(19) United States
(12) Patent Application Publication
(10) Pub. No.: US 2026/0132188 A1
(43) Pub. Date: May 14, 2026

(54) Antibody Cocktails for the Treatment of Viral Infections

(71) Applicant: Longhorn Vaccines and Diagnostics, LLC, Bethesda, MD (US)

(72) Inventors: Jeffrey D. Fischer, Washington, DC (US); Clara J. Sei, Germantown, MD (US); Gerald W. Fischer, Bethesda, MD (US)

(73) Assignee: Longhorn Vaccines and Diagnostics, LLC, Bethesda, MD (US)

(21) Appl. No.: 19/435,922

(22) Filed: Dec. 30, 2025

Related U.S. Application Data
(63) Continuation-in-part of application No. 19/357,162, filed on Oct. 14, 2025.
(63) Continuation-in-part of application No. 19/182,300, filed on Apr. 17, 2025.
(60) Provisional application No. 63/635,703, filed on Apr. 18, 2024.
(60) Provisional application No. 63/662,027, filed on Jun. 20, 2024.
(60) Provisional application No. 63/706,770, filed on Oct. 14, 2024.
(60) Provisional application No. 63/709,551, filed on Oct. 21, 2024.
(60) Provisional application No. 63/738,652, filed on Dec. 24, 2024.
(60) Provisional application No. 63/740,742, filed on Dec. 31, 2024.
(60) Provisional application No. 63/744,226, filed on Jan. 11, 2025.
(60) Provisional application No. 63/877,254, filed on Sep. 7, 2025.

(51) Int. Cl. C07K 16/108; A61K 39/00; C07K 16/104

(52) U.S. Cl. CPC C07K 16/108; C07K 16/104; A61K 2039/507

(57) ABSTRACT

The invention is directed to cocktails of different antibodies and methods of treating a patient. Antibodies of the collection are specifically reactive to different viral serotypes or species. Cocktails may comprise monoclonal antibodies with one or more having an extended half-life. For treating or preventing viral infections, cocktails may comprise antibodies against conserved regions of HA, NA and M proteins of influenza virus and/or conserved regions of S, M, E, P, N or NS proteins of coronavirus. Monoclonal antibody combinations enhance phagocytosis and killing of viruses by phagocytic cells, and enhance clearance of the infecting virus from the blood.

### REFERENCE TO RELATED APPLICATIONS

This application claims priority to U.S. Provisional Application No. 63/740,742 filed Dec. 31, 2024, U.S. Provisional Application No. 63/744,226 filed Jan. 11, 2025, and U.S. Provisional Application No. 63/877,254 filed Sep. 7, 2025, and U.S. patent application Ser. No. 19/357,162, filed Oct. 14, 2025, which claims priority to U.S. Provisional Application No. 63/738,652 filed Dec. 24, 2024, U.S. Provisional Application No. 63/709,551 filed Oct. 21, 2024, and U.S. Provisional Application No. 63/706,770 filed Oct. 14, 2024, and U.S. application Ser. No. 19/182,300 filed Apr. 17, 2025, which claims priority to U.S. Provisional Application No. 63/662,027 filed Jun. 20, 2024, and U.S. Provisional Application No. 63/635,703 filed Apr. 18, 2024, the entirety of each of which is incorporated.

### SEQUENCE LISTING

The instant application contains a Sequence Listing which has been submitted electronically in XML format and is hereby incorporated by reference in its entirety. Said XML copy, created on Dec. 29, 2025, is named 3022_063_US_SL.xml and is 126,054 bytes in size.

### BACKGROUND

### 1. Field of the Invention

The present invention is directed to cocktails of different antibodies and methods for preventing and treating viral diseases and/or disorder associated with infections by viral microorganisms. In particular, antibody cocktails of the disclosure contain multiple antibodies with extended half-lives that are specific, bi-specific, or multi-specific to different conserved epitopes for the prevention and treatment of seasonal and pandemic infections.

### 2. Description of the Background

In virus classification, influenza viruses are negative sense, single strand RNA viruses. The genera of influenza viruses currently comprise the Orthomyxoviridae Family: Influenza virus A, Influenza virus B, Influenza virus C, and Influenza virus D. Each of these genera contains a single species of influenza virus.

The genus Influenza virus A consists of a single species, influenza A virus, which includes all of the influenza virus strains currently circulating among humans. The different types of Influenza A are based on two proteins on the surface of the virus: hemagglutinin (H or HA) and neuraminidase (N or NA). There are at least 18 different hemagglutinin subtypes and at least 11 different neuraminidase subtypes (H1 through H18 and N1 through N11, respectively). These include, for example, but not limited to, H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7 serotypes. Influenza A subtypes (e.g., H1N1) can be further broken down into different genetic clades (e.g., 6B.1) and sub-clades (e.g., 6B.1A). Although clades and sub-clades are genetically different they are not necessarily antigenically different. There are potentially many more influenza A subtype combinations given the propensity for virus reassortment. Reassortment is a process by which influenza viruses swap gene segments. Reassortment can occur when two different influenza viruses infect the same host at the same time and swap genetic information.

The genus Influenza virus B consists of a single species, influenza B virus, of which there is currently only one known serotype, but further classified into two lineages: B/Yamagata and B/Victoria. Influenza B virus is almost exclusively a human pathogen but is significantly less common and less genetically diverse than influenza A strains. Because of this limited genetic diversity, most humans acquire a certain degree of immunity to influenza B virus at an early age; however, the mutation frequency of the virus is sufficiently high enough to prevent lasting immunity by most humans, but not high enough to permit pandemic infection by influenza B virus across human populations. Similar to influenza A viruses, influenza B viruses can then be further classified into specific clades (e.g., V1A) and sub-clades (e.g., V1A1).

The genus Influenza virus C also consists of a single species, denoted influenza C virus, of which there is also currently only one known serotype. This serotype is known to infect both primates and porcine, and while human infections of influenza C virus are rare, the resulting illness can be mild to severe. Epidemics of influenza C virus are not uncommon in exposed populations, however, due to its rapid transmissibility in humans having close contact.

A fourth family of influenza viruses was identified in 2016-Influenza D, which was first isolated in 2011. Hemagglutinin (HA) and neuraminidase (NA) are the two large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins are targets for antiviral drugs. Furthermore, they are antigens to which antibodies can be raised. Influenza A viruses are classified into subtypes based on antibody responses to HA and NA. These different types of HA and NA form the basis of the H and N distinctions in, for example, H5N1. There are 18 HA and 11 NA subtypes known, but only HA 1, 2 and 3, and NA 1 and 2 are commonly found in humans. Influenza A virus, in particular, has many different serotypes, upwards of 144 possible “HN” serotypes based on variations within these two proteins alone. Only a small number of these combinations are believed to be circulating within susceptible populations at any given time.

Influenza viruses are etiologic agents for a contagious respiratory illness (commonly referred to as the flu) that primarily affects humans and other vertebrates. Influenza is highly infectious and an acute respiratory disease that has plagued the human race since ancient times. Infection is characterized by recurrent annual epidemics and periodic major worldwide pandemics. Influenza virus infection can cause mild to severe illness and can even lead to death. Every year in the United States, 5 to 20 percent of the population, on average, contracts the flu with more than 200,000 hospitalizations from complications and over 36,000 deaths. Because of the high disease-related morbidity and mortality, direct and indirect social economic impacts of influenza are enormous. Four pandemics occurred in the last century, together causing tens of millions of deaths worldwide.

Influenza virus spreads from avian to human hosts through close contact. The genome of avian influenza virus (AIV) consists of eight segments of single-stranded, negative-sense RNA that codes for the proteins designated as: PB2, PB1, PB1-F2, PA, HA, NP, NA, M1, M2, M2e, NS1, and NS2/NEP. These proteins include Hemagglutinin (HA), a surface glycoprotein that attaches the virus to cells for entry; Neuraminidase (NA), surface glycoprotein that releases the virus from cells; Matrix protein 1 (M1), a protein that forms a matrix around the virion core; Matrix protein 2 (M2 and M2e), transmembrane proteins with a proton channel; Nucleoprotein (NP), a protein that coats the viral RNA segments; Polymerase basic 1 (PB1), a subunit of the heterotrimeric RNA-dependent RNA polymerase; Polymerase basic 2 (PB2), a subunit of the heterotrimeric RNA-dependent RNA polymerase; Polymerase acidic (PA), a subunit of the heterotrimeric RNA-dependent RNA polymerase; Nonstructural protein 1 (NS1), a protein encoded by the influenza A genome; and Nonstructural protein 2 (NS2), also known as the nuclear export protein (NEP).

Coronaviruses are a group of RNA viruses that cause diseases in mammals and birds. Coronaviruses are viruses in the subfamily Orthocoronavirinae in the family Coronaviridae, in the order Nidovirales and contains proteins referred to as S (spike), E (envelope), M (matrix), N (nucleocapsid), and nonstructural proteins (NS) such as P (polymerase). Coronaviruses are enveloped viruses with a positive-sense single-stranded RNA genome and with a nucleocapsid of helical symmetry. The genomic size of coronaviruses ranges from approximately 26 to 32 kilobases, the largest for an RNA virus. The name “coronavirus” is derived from the Latin corona, meaning crown or halo, which refers to the characteristic appearance of the virus particles (virions): they have a fringe reminiscent of a royal crown or of the solar corona. In humans, the viruses cause respiratory infections including what is referred to as the common cold. Coronavirus is also the etiological agent of SARS, MERS, and the 2019-20-Wuhan outbreak.

Influenza virus and coronavirus spread from host to host through coughing or sneezing. Airborne droplets are the primary transmission vectors between individuals. In humans, the virus typically spreads directly from person to person, although persons can also be infected from indirect contact with surfaces harboring the virus. Infected adults become infectious to others beginning as little as one day before primary symptoms of the disease develop. Thereafter, these persons remain infectious for up to 5 days or more after. Uncomplicated illness is often characterized by an abrupt onset of constitutional and respiratory symptoms, including fever, myalgia, headache, malaise, nonproductive cough, sore throat, rhinitis, or a combination of one or more of these symptoms.

Animal workers are typically the first human hosts of a new strain of virus. These new strains emerge as a result of “antigenic drift.” Antigenic drift is caused by mutations within antigenic (i.e., immunity stimulating) portions of viral proteins within viral subtypes circulating in host populations that alter the host's ability to recognize and defend effectively against the infecting virus, even when the virus has been circulating in the community for several years. The antigenic drift that diminishes existing immunity in a host population generally occurs within so-called immunodominant antigens or regions. Immunodominant antigens are those antigens belonging to a pathogen that are the most-easily and most-quickly recognized by the host immune system and, consequently, account for the vast majority of immune responses to the invading pathogen. Typically, immunodominant antigens exist within regions of the pathogen that are most exposed to the environment, i.e., are on the external surfaces or on protruding elements of the pathogen, and so are most readily accessible to the host immune system.

In the case of influenza, the immunodominant HA and NA proteins protrude from the central capsid of the viral particle, and the spike protein of coronavirus, so they tend to interact most strongly with the host's internal environment and dominate the host immune response. Mutations occurring in the microbial genome that protect the microbe from the host immune system, these mutations are most readily found to affect the immunodominant antigens.

Non-immunodominant antigens are those that are capable of raising a host immune response but account for only a small amount of the total immune response. This is thought to happen because the non-immunodominant antigens are at least partially shielded from the host immune system, as in the case of an antigen that is located in a cleft or fold of the microbial surface or is surrounded by protruding elements of the microbe. In the case of influenza and coronaviris, non-immunodominant antigens occurring near the capsid surface are shielded from the host immune system by the immunodominant HA and NA and spike proteins of coronavirus, protruding from the surface. Non-immunodominant antigens tend to show less mutation in response to host immune pressure than do immunodominant antigens.

The United States Center for Disease Control and Prevention (CDC) and the leading authorities on disease prevention in the world recommend the single best way of preventing a viral respiratory infection in humans is through regular vaccinations. Conventional flu vaccines typically target the immunodominant proteins, HA and NA antigens, and coronavirus vaccines target spike protein. However, these vaccines have not been universally protective or 100 percent effective at preventing the disease. Antigenic shift prevents flu vaccines from being universally protective or from maintaining effectiveness over many years. The ineffectiveness of conventional flu vaccines may also be due, in part, to antigenic drift and the resulting variation within antigenic portions of the HA, NA, and spike proteins most commonly recognized by the immune system (i.e., immunodominant antigens). As a result, many humans may find themselves susceptible to the viral virus without an effective method of treatment available since the virus is constantly improving its resistance to current treatments. This scenario is particularly concerning with respect to the H5N1 virus, which is highly virulent but for which there is currently no widely available commercial vaccine to immunize and protect susceptible human populations.

Currently, flu vaccines are reformulated each year due to the yearly emergence of new strains, and generally induce limited immunity. In addition, to achieve a protective immune response, some vaccines are administered with high doses of antigen, which can be detrimental. This is particularly true for H5N1 vaccines. In addition, influenza vaccines, including H5N1 vaccines, typically present epitopes in the same order as the epitopes are found in nature, generally presenting as whole-viral proteins; consequently, relatively large amounts of protein are required to make an effective vaccine. As a result, each administration includes, in addition to added risk, an increased cost associated with the dose amount, and there is increased difficulty in manufacturing enough doses to vaccinate the general public. Further, the use of larger proteins elevates the risk of undesirable immune responses in the recipient host.

Thus, there is a strong need to provide or improve products and treatments to provide broader protection against not only existing but new and emerging strains and serotypes, to reduce unwanted and undesirable side effects, and to decrease costs in the battle against influenza virus infections.

### SUMMARY OF THE INVENTION

The present invention overcomes the problems and disadvantages associated with current strategies and designs and provides new tools and methods for treating or preventing infection and enhancing the immune system of a patient.

One embodiment of the invention is directed to collections of different antibodies that are reactive to two or more of the epitopes of different influenza virus and/or coronavirus proteins. Those proteins include PB2, PB1, PB1-F2, PA, HA, NP, NA, M1, M2, M2e, NS1, and NS2/NEP proteins of influenza virus. Proteins that contribute to the overall structure of all coronaviruses are the spike(S), envelope (E), membrane (M), and nucleocapsid (N), and also internal proteins such as polymerase (P). The immunodominant protein is believed to be the spike protein as it interacts with the host's internal environment and dominates the host immune response.

The sequences of a number of the epitopes associated with these proteins are disclosed herein. The different epitopes may be of different species or serotypes of the same viral microorganism, to different species or serotypes of different viruses, or to combinations thereof. Preferably the antibody comprises one or more of the isotypes IgG (including IgG1, IgG2a, IgG2b, IgG3), IgA, IgD, IgE, and IgM or fragments (e.g., Fc, Fhv, Fab) or combinations thereof. Antibodies may also be formulated into compositions for treatment of a subject containing pharmaceutically acceptable carriers or other substances. Preferably the antibody is a polyclonal, monoclonal, or a partly or fully human antibody. Preferably the antibodies are monoclonal antibodies, but may be bi-specific, multi-specific, or polyclonal as desired. Preferably the antibody is fully or partly human. The multi-specific antibody may include a neuraminidase inhibitor. Preferably monoclonal antibodies of the collection have a normal half-life or have been altered to have an extended half-life on the order of 1.5 times greater, 2 times greater, 4 times greater, 8 times greater, 10 times greater, 20 times greater, or greater than the half-life of an unaltered antibody. There are a number of strategies to extend the half-life of antibodies, namely, pegylation, glycosylation, recombinant engineering, and crystallizable of the Fc region. Modifications are preferably through recombinant engineering such as YTE modifications, LS variants, or REW mutations. Preferably, antibody collections comprise monoclonal antibodies with extended half-lives that react with one, two and more preferably three of the proteins selected from influenza proteins NA, HA. M1, M2 and M2e, and/or one or more of the coronavirus proteins selected from S, E, M, N, and P.

Another embodiment of the invention is directed to individual monoclonal antibodies that are specifically reactive to at least two different conserved regions of one or more different viral proteins. These antibodies are referred to as multi-specific (e.g., bi-specific, tri-specific, etc.). Preferably the multi-specific antibodies have been altered to have an extended half-life as compared to an unaltered form of the monoclonal antibody. Preferably, the altered form of the antibody is recombinantly modified with YTE, LS, or REW modifications. Preferably the multi-specific antibodies are IgG or IgM, and partly or fully human or humanized. Preferably the different viral proteins are selected from the group consisting of influenza virus hemagglutinin protein (HA), neuraminidase protein (NA), matrix protein M1, matrix protein M2, and matrix protein M2e, and/or the group consisting of coronavirus spike protein(S), nucleocapsid protein (NC), matrix protein (M), envelope protein (E), and polymerase protein (P). Preferably the antibodies are specifically reactive to epitopes of a coronavirus protein and epitopes of an influenza virus protein such as, for example, a conserved region of a coronavirus polymerase protein and a conserved region of an influenza virus neuraminidase protein.

Another embodiment of the invention is directed to methods of treatment comprising administering a collection of antibodies as described herein to a subject infected or at risk of being infected with Influenza and/or coronavirus. Alternatively, or in addition to the collection, such subjects may be administered compositions that promote the immune system's ability to respond to a viral infection. Preferably the subject is a mammal that, after administration, clears the virus from the subject's system. Preferably administration generates an immune response within the host that promotes neutralization of the virus.

Another embodiment of the invention is directed to hybridomas that express monoclonal antibodies as disclosed herein.

Other embodiments and advantages of the invention are set forth in part in the description, which follows, and in part, may be obvious from this description, or may be learned from the practice of the invention.

### DESCRIPTION OF THE INVENTION

Influenza and Corona viruses are etiologic agents for a contagious respiratory illness that primarily affects humans and other vertebrates. Uncomplicated influenza illness is often characterized by an abrupt onset of constitutional and respiratory symptoms, including fever, myalgia, headache, malaise, nonproductive cough, sore throat, rhinitis, or a combination of one or more of these symptoms. More complicated forms of infection can cause severe illness and can even lead to death. Every year in the United States, on average, 5 percent to 20 percent of the population contract the Flu with more than 200,000 hospitalizations.

Both Influenza and Corona viruses spread from host to host through coughing or sneezing, with airborne droplets as the primary vectors of the disease. In humans, the virus usually spreads directly from person to person, although subjects can sometimes become infected by indirect contact of surfaces harboring the virus, and then touching their mouth or nose. Most healthy adults may be able to infect others beginning as much as a day before primary symptoms of the disease develop and remain contagious for up to five days after becoming infected.

Inactivated viral vaccines are now available worldwide and are utilized especially in high-risk groups such as infants, the elderly, those without adequate health care, and immunocompromised individuals. The viruses comprising the influenza vaccine are grown in eggs, inactivated by chemical means, and thereafter purified. The vaccines are usually bivalent or trivalent, containing representative influenza A viruses (H1N1 and H3N2), influenza B strains and coronavirus SP proteins, regularly updated to maintain efficacy. These viral vaccines are reformulated annually due to the emergence of new strains. During inter-pandemic periods, vaccine formulation usually takes a minimum of eight months before an updated viral vaccine is ready for market. Historically, however, viral pandemics are spread to most continents within four to six months, and future viral pandemics are likely to spread even faster due to increased international travel. It is therefore inevitable that an effective vaccine made by conventional means will be unavailable or in very short supply during the first wave of any future widespread outbreak or pandemic.

It has been surprisingly discovered that collections of antibodies, targeted against different regions of a virus, preferably conserved regions, when administered to a subject block the life cycle of the virus, including virus multiplication and/or development of virus in the host cell. Antibody collections as disclosed herein promote the cell killing mechanisms of the immune system including, but not limited to phagocytosis, apoptosis, macrophage and natural-killer cell activation, cytokine and T-cell modulation and complement-initiated cell lysis. Without being limited by theory, the cocktail is believed to inhibit entry of viral particles, and/or exit of viral particles from host cells, and also replication of the viral genome, thereby neutralizing infection of the virus and/or the infectability of the host to others. As collection of antibodies as disclosed herein are less susceptible or more resistant to variations in antigenic shift and drift compared to conventional vaccines, administration of collections of antibodies reduces or eliminate the need for repeated vaccinations of susceptible patient populations against potential outbreaks of infection from new viral isolates. Thus, the invention further provides methods of preventing or controlling an outbreak of viral infection in a selected mammalian population. The method allows health care professionals to take immediate steps to control a new and emerging viral infection in a population, which can thereby prevent, reduce, lessen, alleviate, control, or delay the outbreak of such an infection in the general population including susceptible or at-risk members. The antibodies of the invention function almost immediately to shut down, not only the virus infecting the host, but also to shut down or at least substantially reduces the ability of that host to infect others.

Preferably antibodies of the collection bind to different viral antigens or epitopes such as any two or more regions of the proteins PB2, PB1, PB1-F2, PA, HA, NP, NA, M1, M2, M2e, NS1, and NS2/NEP, and/or coronavirus S, E, M, N, and P proteins. Various of these regions and combinations of regions are disclosed in Example 1. Epitopes are regions obtained or derived from a protein or peptide of a pathogen that elicit a robust immunological response when administered to a mammal or an animal. Preferably the epitopes are derived from conserved regions of proteins of viral particles. The virus may be of the same type, but of a different strain, serotype or species or other relation. The antibodies may also be reactive to composite epitopes of this disclosure which are single epitopes that represent a composite of two conserved regions or two related epitopes (e.g., for Influenza HA and HA; NA and NA, M and M2e, etc.: for coronavirus S and S, M and M, etc.), or other conserved regions.

Conserved regions are sequences of the virus that are very similar between different strains or serotypes of the same virus. Differences between the conserved regions of different strains and/or serotypes of a virus are preferably less five amino acids between two known conserved regions, less four amino acids between two known conserved regions, less three amino acids between two known conserved regions, less two amino acids between two known conserved regions, and one amino acid difference between two known conserved regions.

An antibody or antibody collection that reacts with various Influenza viral protein epitopes to include, but not limited to antibodies that react with one or more epitopes of the M1 protein plus one or more epitopes of the HA and/or NA proteins; one or more epitopes of the M2 protein plus one or more epitopes of the HA and/or NA proteins; and/or one or more epitopes of the M2e protein plus one or more epitopes of the HA and/or NA proteins. An antibody or antibody collection that reacts with various coronavirus protein epitopes including, but not limited to antibodies that react with one or more epitopes of the P protein plus one or more epitopes of the M or E proteins; one or more epitopes of the S protein. Epitopes may be from conserved regions of these proteins and may be composite epitopes. Composite epitopes, are epitopes that are engineered and artificially synthesized. Composite epitopes are created from two or more different but otherwise very similar epitopes, which are merged or otherwise artificially created in the form of a single new epitope which carries the same immunological properties as the original two epitopes. Composite epitopes are preferably derived from highly conserved regions of slightly different sequences of the same epitope of different strains or serotypes of the virus. Antigens that contain one or more composite epitopes are referred to as composite antigens. Composite epitopes are preferably constructed of conserved regions of different epitopes (e.g., M, HA, and NA of influenza virus, or P, E, M, S of Coronavirus). Preferably, the composite antigen, when exposed to the immune system of a mammal or an animal, is capable of simultaneously generating an immunological response to each of the constituent epitopes of the composite and preferably to the same or a greater degree (e.g., as measurable from a cellular or humoral response to an identified pathogen) than the individual epitopes.

Preferred combinations of MABs include combinations that contain at least one MAB to an influenza NA protein, combinations that contain at least one MAB to an influenza M (e.g., M1, M2e) protein, combinations that contain at least one MAB to a coronavirus P protein, and combinations that contain at least one MAB to a coronavirus M or E protein.

Individual antibodies that are reactive against one or more different peptide epitopes may be specific, bi-specific, or multi-specific, or monoclonal or polyclonal and may be derived from any mammal such as, for example but not limited to, mouse, rabbit, goat, pig, guinea pig, rat and preferably human. Multi-specific antibodies (e.g., bi-specific, tri-specific, etc.) preferably are reactive to conserved regions or epitopes of different parts of a virus or of different viruses. For example, bi-specific antibodies may be reactive to both an epitope of an influenza virus protein and an epitope of a coronavirus protein.

Polyclonal antibodies may be collected from the serum of infected or carrier mammals (e.g., typically human, although equine, bovine, porcine, ovine, or caprine may also be utilized) and preserved for subsequent administration to patients with existing infections. Monoclonal antibodies may be of one or more of the classes IgA, IgD, IgE, IgG, or IgM, containing alpha, delta, epsilon, gamma or mu heavy chains and kappa or lambda light chains, or any combination heavy and light chains including effective fractions thereof, such as, for example, single-chain antibodies, isolated variable regions, isolated Fab or Fc fragments, isolated complement determining regions (CDRs), and isolated antibody monomers. Monoclonal antibodies may be created or derived from human or non-human cells and, if non-human cells, they may be chimeric MABs or humanized. Non-human antibodies are preferably humanized by modifying the amino acid sequence of the heavy and/or light chains of peptides to be similar to human variants, or genetic manipulation or recombination of the non-coding structures from non-human to human origins.

Preferably one or more or all of the antibodies of a collection of the invention are modified to have extended circulating half-lives in serum as compared to unmodified antibodies. Modifications to extend antibody half-lives include modification of the Fc domain to promote FcRn interaction. YTE mutation at the CH2—CH3 interface which constitutes contact residues for FcRn binding. Modifications are preferably through recombinant engineering such as YTE modifications (specific amino acid changes at M252Y, S254T, T256E in the Fc region of an antibody), LS variants (specific amino acid changes at M428L/N434S in the Fc region of an antibody), or REW mutations at Q311R/M428E/N434W of the Fc region. The YTE mutation is highly solvent exposed where it can constitute a discontinuous B cell epitope. Modifications also include increasing the hydrodynamic radius of the protein, e.g., by hyperglycosylation, coupling to polyethylene glycol (PEG) chains or other hydrophilic polymers, and fusion to PEG-mimetic polypeptide chains or long circulation plasma protein and fragments thereof. Unmodified antibodies have varying half-lives depending on the isotype, with an average of about three weeks. Modified antibodies can have up to and including 150 percent greater circulating half-life, 200 percent greater circulating half-life, 300 percent greater circulating half-life, 400 percent greater circulating half-life, 500 percent greater circulating half-life, or more. These extended half-life MABs remain in the system for longer periods of time than conventional antibodies and block cell entry or cell exit can be combined with MABs that impede intracellular replication to provide a neutralizing MAB cocktail across more than one strain of the virus.

Antibody collections of this disclosure may include pharmaceutically acceptable carriers and/or adjuvants. Pharmaceutically acceptable carriers include, for example, a chemical agent, diluent, or excipient comprises water, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, or a combination thereof. Other examples include, for example, alum, oil in water emulsion, amino acids, proteins, carbohydrates, Freund's, liposome, saponin, lipid A, squalene, liposomes adsorbed to aluminum hydroxide, liposomes containing QS21 saponin, liposomes containing QS21 saponin and adsorbed to aluminum hydroxide, liposomes containing saturated phospholipids, cholesterol, and/or monophosphoryl, ALFQ, ALFA, AS01, and/or modifications or derivatives thereof.

Another embodiment of the invention is directed to the administration of bi-specific or multi-specific MABs or multiple different antibodies as disclosed herein (e.g., polyclonal, monoclonal or fractions such as Fab fragments, amino acid sequences of the variable binding antibody regions, single chains, etc.), antibody collections, for the treatment of a virus infection. Administration of antibodies for prevention or treatment of infections, whether polyclonal or monoclonal, may be through a variety of available mechanisms including, but not limited to inhalation, subcutaneous (SQ), intravenous (IV), intraperitoneal (IP), intradermal (ID), intramuscular (IM) injection. Administration may be at regular or irregular intervals, or as a single dose.

Antibody collections may be further combined with conventional antibodies (polyclonal, monoclonal or fractions such as Fab fragments, single chains, etc.) into an antibody cocktail for the treatment and/or prevention of an infections. Combinations can include two, three, four, five or many more different antibody combinations with each directed to a different epitope of a peptide sequence of virus proteins.

The invention further comprises hybridoma cell lines created from the fusion of antibody producing cells with human or other cell lines for the generation of monoclonal antibodies of the invention.

Another embodiment of the invention is directed to the prophylactic administration of collections of antibodies as disclosed herein to protect immune compromised patients and health care workers who administer to patients and, in particular, patients with virus infections. At present, a health care professional, or most anyone, who treats or cares for a patient infected with a virus is at extreme risk for acquiring the same infection as those he or she cares for. There is also a substantial risk to all persons within a general health care facility that such a viral infection will be acquired by other health care workers at the facility or visitor who otherwise have no contact or interaction with such patients. With the prophylactic administration of antibodies of the invention to health care workers, they are able to care for and attend to these patients.

Antibodies used alone or in conjunction with other anti-viral therapies will increase the clearance of virions from the blood and/or other tissues, or inactivate substances that impede immunity as measured by a more rapid reduction of symptoms, more rapid time to smear negativity and improved weight gain and general health. In addition, treatment provides an effective reduction in the severity of symptoms, the generation of immunity to Influenza, and/or the reduction of infective period of time. Preferably the patient is administered an effective amount, combined with anti-virals, antibodies to prevent or overcome an infection alone or as adjunctive therapy.

Treatment of subjects may be combined with antivirals, cytokines and additional substances (e.g., substances that inhibit protein or nucleic acid synthesis, substances that injure existing or developing membranes or other virus structures, substances that inhibit synthesis of essential metabolites of the virus), or one or more substances that attack the virus itself. Effective amounts of such substances are expected to be less than the manufacturer recommended amount or higher dose, but for short periods of time (e.g., about one hour, about 4 hours, about 6 hours, less than one or two days).

Although the invention is generally described in reference to human viral infection, as is clear to those skilled in the art the compositions including many of the compositions, tools and methodology is generally and specifically applicable to the treatment and prevention of many viral infection in many other subjects (e.g., cats, dogs, pets, horses, cattle, pigs, avian species, farm animals, etc.).

The following examples illustrate embodiments of the invention but should not be viewed as limiting the scope of the invention.

### EXAMPLES

### Example 1 Peptides and Sequences

The following is a list of exemplary peptide sequences and composite peptide sequences that are responsive to antibodies of the invention and/or antibodies are created that are responsive to and also bind to these sequences.

Influenza Virus Sequences

| | |
|---|---|
| SEQ ID NO 1 | DWSGYSGSFVQHPELTGLD (N1 sequence; H1 N5) |
| SEQ ID NO 2 | ETPIRNE (M2e epitope) |
| SEQ ID NO 3 | FVIREPFISCSHLEC |
| SEQ ID NO 4 | GNFIAP (HA epitope; Pep 1) |
| SEQ ID NO 5 | GNLIAP (HA epitope; Pep 2) |
| SEQ ID NO 6 | GNLFIAP (composite sequence of SEQ ID NOs 4 and 5; Pep 3) |
| SEQ ID NO 7 | GNLIFAP (composite sequence of SEQ ID NOs 4 and 5) |
| SEQ ID NO 8 | HYEECSCY (NA epitope; Pep 10) |
| SEQ ID NO 9 | LLTEVETPIR (highly conserved region M1/M2e) |
| SEQ ID NO 10 | LLTEVETPIRN (highly conserved region M1/M2e) |
| SEQ ID NO 11 | LLTEVETPIRNE (highly conserved region M1/M2e) |
| SEQ ID NO 12 | DWSGYSGSFVQHPELTGL (N1 sequence; H1 N5) |
| SEQ ID NO 13 | EVETPIRNE (highly conserved region M1/M2e) |
| SEQ ID NO 14 | FLLPEDETPIRNEWGLLTDDETPIRQYIKANSKFIGITE |
| SEQ ID NO 15 | GNLFIAPGNLFIAPHYEECSCYHYEECSCYQYIKANSKFIGITEHY |
| | EECSCYTPIRNETPIRNE (composite sequence of SEQ ID NO 4 HA, |
| | SEQ ID NO 4 HA, SEQ ID NO 8 NA, SEQ ID NO 8 NA, T-cell epitope, |
| | SEQ ID NO 4 NA, SEQ ID NO 2 M2e, SEQ ID NO 2 M2e) |
| SEQ ID NO 16 | GNLFIAPGNLFIAPQYIKANSKFIGITEGNLFIAP (composite of SEQ |
| | ID NO 6 HA, SEQ ID NO 6 HA, SEQ ID NO 61 T-cell epitope, and SEQ |
| | ID NO 6) |
| SEQ ID NO 17 | HYEECSCYDWSGYSGSFVQHPELTGLHYEECSCYQYIKAN |
| | SKFIGITE (composite sequence of SEQ ID NO 8 NA, SEQ ID NO 1 N1 |
| | sequence H1 N5, SEQ ID NO 8 NA, SEQ ID NO 61 T-cell epitope. |
| SEQ ID NO 18 | ITGFAPFSKDNSIRLSAGGDIWVTREPYVSCDP |
| SEQ ID NO 19 | IWGIHHP (HA epitope) |
| SEQ ID NO 20 | IWGVHHP (HA epitope) |
| SEQ ID NO 21 | IWGVIHHP (composite of SEQ ID NOs. 19 and 20, HA) |
| SEQ ID NO 22 | IWGIVHHP (composite of SEQ ID NOs. 19 and 20, HA) |
| SEQ ID NO 23 | KSCINRCFYVELIRGR (N3 conserved epitope) |
| SEQ ID NO 24 | LLTEVETPIRNESLLTEVETPIRNEWG (M2e epitope) |
| SEQ ID NO 25 | LLTEVETPIRNEW (M2e epitope) |
| SEQ ID NO 26 | LLTEVETPIRNEWG (M2e epitope) |
| SEQ ID NO 27 | LTEVETPIRNE (M2e epitope) |
| SEQ ID NO 28 | LTEVETPIRNEW (M2e epitope) |
| SEQ ID NO 29 | LTEVETPIRNEWG (M2e epitope) |
| SEQ ID NO 30 | MSLLTEVET (M2e epitope) |
| SEQ ID NO 31 | MSLLTEVETP (M2e epitope) |
| SEQ ID NO 32 | MSLLTEVETPI (M2e epitope) |
| SEQ ID NO 33 | MSLLTEVETPIR (M2e epitope) |
| SEQ ID NO 34 | MSLLTEVETPIRN (M2e epitope) |
| SEQ ID NO 35 | MSLLTEVETPIRNE (M2e epitopes) |
| SEQ ID NO 36 | MSLLTEVETPIRNETPIRNE (M2e epitope) |
| SEQ ID NO 37 | MSLLTEVETPIRNEW (M2e epitope) |
| SEQ ID NO 38 | MSLLTEVETPIRNEWG (M2e epitope) |
| SEQ ID NO 39 | MSLLTEVETPIRNEWGCRCNDSSD (M2e epitope) |
| SEQ ID NO 40 | SLLTEVET (M2e epitope) |
| SEQ ID NO 41 | SLLTEVETPIR (M2e epitope) |
| SEQ ID NO 42 | SLLTEVETPIRNE (M2e epitope) |
| SEQ ID NO 43 | SLLTEVETPIRNEW (M2e epitope) |
| SEQ ID NO 44 | SLLTEVETPIRNEWG (M2e epitope) |
| SEQ ID NO 45 | SLLTEVETPIRNEWGTPIRNE (M2e epitope) |
| SEQ ID NO 46 | SLLTEVETPIRNEWGTPIRNETPIRNE (M2e epitope) |
| SEQ ID NO 47 | SLLTEVETPIRNEWGTPIRNETPIRNETPIRNE (M2e epitopes) |
| SEQ ID NO 48 | SLLTEVETPIRNEWGLLTEVETPIR (M1/M2e conserved region) |
| SEQ ID NO 49 | TEVETPIRNE (M2e epitope) |
| SEQ ID NO 50 | TPIRNE (M2e epitope) |
| SEQ ID NO 51 | VETPIRNE (M2e epitope) |
| SEQ ID NO 52 | VTREPYVSCDPKSCINRCFYVELIRGRVTREPYVSCDPWYIK |
| | ANSKFIGITE |
| SEQ ID NO 53 | WGIHHP (HA conserved region; Pep 05) |
| SEQ ID NO 54 | WGVHHP (HA conserved region; Pep 04) |
| SEQ ID NO 55 | WGVIHHP (composite of SEQ ID NOs 53 and 54; Pep 06) |
| SEQ ID NO 56 | WGIVHHP (composite of SEQ ID NOs 53 and 54; Pep 07) |
| SEQ ID NO 57 | YIWGIHHP (HA conserved region) |
| SEQ ID NO 58 | YIWGVHHP (HA conserved region) |
| SEQ ID NO 59 | YIWGVIHHP (composite of SEQ ID NOs 57 and 58) |
| SEQ ID NO 60 | YIWGIVHHP (composite of SEQ ID NOs 57 and 58) |
| SEQ ID NO 61 | QYIKANSKFIGITE (T cell epitope) |
| SEQ ID NO 62 | PIRNEWGCRCNDSSD (M2e epitope) |
| SEQ ID NO 63 | GNLFIAPWGVIHHPHYEECSCY (underlined sequences are epitopes |
| | HA {composite} (SEQ ID NO 6) and NA (SEQ ID NO 8), respectively, |
| | with middle as SEQ ID NO 55, of Influenza A; Pep 11) |
| SEQ ID NO 64 | CAGAGNFIAP (CAGA domain, HA) |
| SEQ ID NO 65 | CAGAGNLIAP (CAGA domain, HA) |
| SEQ ID NO 66 | CAGAGNLFIAP (CAGA domain, HA) |
| SEQ ID NO 67 | CAGAWGVHHP (CAGA domain, HA) |
| SEQ ID NO 68 | CAGAWGIHHP (CAGA domain, HA) |
| SEQ ID NO 69 | CAGAWGVIHHP (CAGA domain, HA) |
| SEQ ID NO 70 | CAGAWGIVHHP (CAGA domain, HA) |
| SEQ ID NO 71 | GNLIAPWGVIHHP (CAGA domain, HA) |
| SEQ ID NO 72 | CAGAGNLIAPWGVIHHP (CAGA domain, HA, HA) |
| SEQ ID NO 73 | GNLFIAPWGVIHHP (HA, HA) |
| SEQ ID NO 74 | CAGAGNLFIAPWGVIHHP (CAGA domain, HA, HA) |
| SEQ ID NO 75 | HYEECSCY (NA) |
| SEQ ID NO 76 | CAGAHYEECSCY (CAGA domain, NA) |
| SEQ ID NO 77 | CAGAGNLFIAPWGVIHHPHYEECSCY (CAGA domain, HA, HA, NA) |
| SEQ ID NO 78 | GNLFIAPWGVIHHPGNLFIAPWGVIHHP (HA, HA, HA, HA) |
| SEQ ID NO 79 | CAGAGNLFIAPWGVIHHPGNLFIAPWGVIHHP (CAGA domain, HA, |
| | HA, HA) |
| SEQ ID NO 80 | HYEECSCYGNLFIAPWGVIHHP (NA, HA, HA) |
| SEQ ID NO 81 | GNLFIAPHYEECSCYWGVIHHP (HA, NA, HA) |
| SEQ ID NO 82 | SLLTEVETPIRNEWGLLTEVETPIRQYIKANSKFIGITE (M2e, T-cell |
| | epitope) |
| SEQ ID NO 83 | GNLFIAPGNLFIAPQYIKANSKFIGITEGNLFIAP (HA, HA, T-cell |
| | epitope, HA) |
| SEQ ID NO 84 | HYEECSCYDWSGYSGSFVQHPELTGLHYEECSCYQYIKANSKFIGITE (NA, N1 |
| | sequence H1 N5, |
| SEQ ID NO 85 | VTREPYVSCDPKSCINRCFYVELIRGRVTREPYVSCDPQYIKANSKFIGITE |
| SEQ ID NO 86 | DWSGYSGSFVQHPELTGL (N1 sequence, H1 N5) |
| SEQ ID NO 87 | ITGFAPFSKDNSIRLSAGGDIWVTREPYVSCDP (Largest conserved |
| | region on N2) |
| SEQ ID NO 88 | KSCINRCFYVELIRGR |
| SEQ ID NO 89 | GNLFIAPRYAFA |
| SEQ ID NO 90 | CAGAGNLFIAPRYAFA |
| SEQ ID NO 91 | GNLVVPRYAFA |
| SEQ ID NO 92 | CAGAGNLVVPRYAFA |
| SEQ ID NO 93 | GNLIAPRYAFA |
| SEQ ID NO 94 | CAGAGNLIAPRYAFA |
| SEQ ID NO 95 | GNLVVP |
| SEQ ID NO 96 | CAGAGNLVVP |
| SEQ ID NO 97 | FVIREPFISCSHLEC |
| SEQ ID NO 98 | CAGAFVIREPFISCSHLEC |
| SEQ ID NO 99 | GNLFIAPWGVIHHPHYEECSCY (Pep 11, HA, HA, NA) |
| SEQ ID NO 100 | GNLFIAPWGVIHHPHYEECSCYQYIKANSKFIGITE |
| | (Pep 11 with C terminal T cell epitope = Pep 63, HA, HA, NA, T-cell |
| | epitope) |
| SEQ ID NO 101 | QYIKANSKFIGITEGNLFIAPWGVIHHPHYEECSCY |
| | (Pep 11 with N terminal T cell epitope = Pep 64, T-cell epitope, HA, HA, |
| | NA) |
| SEQ ID NO 102 | HVEECSY (N1 and N2) |
| SEQ ID NO 103 | WFIHHP (H5) |
| SEQ ID NO 104 | DLWSYNAELLV (stem peptide) |
| SEQ ID NO 105 | DIWTYNAELLV (stem peptide) |
| | HXXXW- matrix peptide common to Flu A and B that constitutes the |
| | main functional element of the M2 channel |
| SEQ ID NO 106 | ARDLICAQKWPWYIWLGFIAGLENQKLIAN |
| | (combination of conserved seqs w/o T cell epitope) |
| SEQ ID NO 107 | ENQKLIANARDLICAQ |
| | (combination of conserved seqs w/o T cell epitope) |
| SEQ ID NO 108 | QYIKANSKFIGITE (Tetanus universal T cell epitope) |
| SEQ ID NO 109 | GNLFIAPWGVIHHPHYEECSCY (composite influenza peptide |
| | comprising HA and NA epitopes) |
| SEQ ID NO 110 | GNLFIAP (Influenza peptide-Pep03 (Hemagglutinin, HA) |
| SEQ ID NO 111 | WGVIHHP (Influenza peptide-Pep06 (Hemagglutinin, HA) |
| SEQ ID NO 112 | HYEECSCY (Influenza peptide-Pep10 (Neuraminidase, NA). |
| SEQ ID NO 113 | ETPIRNE (Influenza peptides-Pep52, M2e). |
| SEQ ID NO 114 | TEVETPIRNE (Influenza peptides-Pep53, M2e). |
| SEQ ID NO 115 | SLLTEVETPIRNEWGLLTEVETPIR (Influenza peptides-Pep57, M2e). |
| SEQ ID NO 116 | GNLFIAPWGVIHHPHYEECSCY (composite influenza peptide |
| | comprising HA and NA epitopes) |
| SEQ ID NO 117 | GNLFIAPWGVIHHPHYEECSCYTEVETPIRNEQYIKANSKFIGITE |
| | (influenza composite epitopes of two HA conserved regions plus NA and |
| | matrix epitopes with T cell epitope) |
| SEQ ID NO 118 | QYIKANSKFIGITEGNLFIAPWGVIHHPHYEECSCYTEVETPIRNE (T cell |
| | epitope with influenza composite epitopes of two HA conserved regions |
| | plus NA and matrix epitopes) |

Coronavirus Sequences

| | |
|---|---|
| SEQ ID NO 119 | YPKCDRA = RNA Polymerase region |
| SEQ ID NO 120 | WDYPKCDRA = RNA Polymerase region |
| SEQ ID NO 121 | SLDQINVTFLDLEYEMKKLEESY w/ QYIKANSKFIGITE (SEQ ID |
| | NO 61) = spike protein w/ tetanus toxoid T cell epitope |
| SEQ ID NO 122 | SLDQINVTFLDLEYEMKKLEESY |
| | (coronavirus spike protein conserved epitope (SP)) |
| SEQ ID NO 123 | SLDQINVTFLDLEYEMKKLEESYQYIKANSKFIGITE |
| | (tetanus toxoid T cell epitope + SP) |
| SEQ ID NO 124 | WDYPKCDRA |
| | (polymerase conserved epitope (POL)) |
| SEQ ID NO 125 | WDYPKCDRAQYIKANSKFIGITE |
| | (POL + tetanus T cell epitope) |
| SEQ ID NO 126 | WDYPKCDRASLDQINVTFLDLEYEMKKLEESYQYIKANSKFIGITE |
| | (Cor POL + SP + Tet) |
| SEQ ID NO 127 | WDYPKCDRATEVETPIRNEHYEECSCYQYIKANSKFIGITE |
| | Cor POL. Flu M2e. Flu NA. Tetanus T cell |

Composite Coronavirus and/or Influenza Peptides

| | |
|---|---|
| SEQ ID NO 128 | SLDQINVTFLDLEYEMKKLEESYQYIKANSKFIGITE |
| | (spike protein epitope with T-cell stimulating epitope) |
| SEQ ID NO 129 | WDYPKCDRA (corona conserved seq |
| SEQ ID NO 130 | YPKCDRA (corona conserved seq-polymerase) |
| SEQ ID NO 131 | ARDLICAQ (conserved SP of Cor, MERS, and SARS) |
| SEQ ID NO 132 | KWPWYIWLGFIAGL (highly conserved cor seq-spike attachment) |
| SEQ ID NO 133 | ENQKLIAN (highly conserved cor seq-spike attachment) |
| SEQ ID NO 134 | ARDLICAQKWPWYIWLGFIAGLENQKLIAN |
| | (combination of conserved seqs w/o T cell epitope) |
| SEQ ID NO 135 | ENQKLIANARDLICAQ |
| | (combination of conserved seqs w/o T cell epitope) |
| SEQ ID NO 136 | WDYPKCDRAENQKLIANARDLICAQ |
| | (combination of conserved seqs w/o T cell epitope) |
| SEQ ID NO 137 | WDYPKCDRAENQKLIANKWPWYIWLGFIAGL |
| | (combination of conserved seqs w/o T cell epitope) |
| SEQ ID NO 138 | ARDLICAQENQKLIANWDYPKCDRAQYIKANSKFIGITE |
| | (combinations of cor conserved seqs w/ T cell epitope) |
| SEQ ID NO 139 | KWPWYIWLGFIAGLWDYPKCDRAQYIKANSKFIGITEARDLIC |
| | AQENQKLIANWDYPKCDRAQYIKANSKFIGITE |
| | (combination of cor conserved seqs w/ T cell epitope) |
| SEQ ID NO 140 | ARDLICAQENQKLIANQYIKANSKFIGITE ARDLICAQENQKLIAN |
| | WDYPKCDRAQYIKANSKFIGITE (combination of cor conserved |
| | seqs w/ T cell epitope) |
| SEQ ID NO 141 | WDYPKCDRATEVETPIRNEHYEECSCYQYIKANSKFIGITE |
| | ARDLICAQENQKLIANWDYPKCDRAQYIKANSKFIGITE |
| | (cor plus Influenza conserved seqs w/ T cell epitope) |
| SEQ ID NO 142 | HYEECSCYWDYPKCDRAVETPIRNEQYIKANSKFIGITE |
| | (cor plus Influenza conserved seqs w/ T cell epitope) |
| SEQ ID NO 143 | ENQKLIANTEVETPIRNEHYEECSCYQYIKANSKFIGITE |
| | (cor plus Influenza conserved seqs w/ T cell epitope) |

Peptide variants of the above sequences include those peptide sequences that contain one or more conservative substitutions of the native sequence. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group, (3) replacement of one or more Val, Ile, Leu, Met, Ala, or Phe residues with one or more residues from the same group; (4) replacement of one or more Lys, Arg. or His residues with one or more residues from the same group; and (5) replacement of one or more Phe. Tyr, Trp, or His residues with one or more residues from the same group. A variant may also, or alternatively, contain non-conservative changes, for example, by substituting one of the amino acid residues from group (1) with an amino acid residue from group (2), group (3), group (4), or group (5). Peptiide variants may also (or alternatively) be modified by, for example, the deletion or addition of amino acids that have minimal to no significant influence on the immunogenicity, secondary structure, and/or hydropathic nature of the peptide, so that the antibody remains substantially equally reactive toward both the native peptide and the varient peptide, and substanially and specifically binds to both sequences.

### Example 2 Three Anti-Flu MAB Treatment (Prophetic)

Three human or humanized hybridomas are created that express monoclonal antibodies each of which is responsive to different conserved region of an Influenza virus protein: one to a conserved region of the NA protein; one to a conserved region of the HA protein; and one to a conserved region of the M1, M2, or M2e protein. These monoclonal antibodies are further modified by recombinant engineering to have at least a two-fold greater half-life as compared to unmodified antibodies. The modified monoclonal antibodies are collected and injected IM or IV one time to a patient with an influenza virus infection and that patient monitored over time. Control IV injections are administered one time to patients which contain only one of the MABs and those patients also monitored over time. The patient receiving an injection of the collection of MABs as compared to patients who received only one of the MABs provides better protection or more rapid recovery from infection.

### Example 3 Three Anti-Flu MAB Treatment (Prophetic)

Hybridomas EA9 (ATCC 127659) and LD9 (ATCC 127662), each expresses anti-HA MABs. Hybridoma NB5 (ATCC 1277140), expresses an anti-NA MAB. Hybridomas GA4 (ATCC 127713), KC7 (ATCC 127660), and CG6 (ATCC 127661), each expresses anti-M MAB. One of EA9 and LD9, GA4, and one of GA4, KC7 and CG6 are humanized and further modified by recombinant engineering to have at least a two-fold greater half-life as compared to unmodified antibodies. The modified monoclonal antibodies are collected and injected IM or IV one time to a patient with an influenza virus infection and that patient monitored over time. Control IV injections are administered one-time to patients which contain only one of the MABs and those patients also monitored over time. The patient receiving an injection of the collection of MABs as compared to patients who received only one of the MABs provides better protection or more rapid recovery from infection.

### Example 4 Three Anti-Coronavirus Mab Treatment (Prophetic)

Three human or humanized hybridomas are created that express monoclonal antibodies responsive to different conserved region of a Coronavirus protein: one to a conserved region of the S protein; one to a conserved region of the M protein; and one to a conserved region of the E protein. These monoclonal antibodies are further modified by recombinant engineering to have at least a two-fold greater half-life as compared to unmodified antibodies. The modified monoclonal antibodies are collected and injected IM or IV one time to a patient with an influenza virus infection and that patient monitored over time. Control IV injections are administered one-time to patients which contain only one of the MABs and those patients also monitored over time. The patient receiving an injection of the collection of MABs as compared to patients who received only one of the MABs provides better protection or more rapid recovery from infection.

Other embodiments and uses of the invention will be apparent to those skilled in the art from consideration of the specification and practice of the invention disclosed herein. All references cited herein, including all publications and U.S. and foreign patents and patent applications, are specifically and entirely incorporated by reference, including U.S. Pat. Nos. 8,821,885; 9,821,047; 9,814,766; 10,004,799; 10,370,437; 11,866,463; 12,403,198; U.S. Pat. No. 12,485,166, US20230201326; US20240050552; US20240123055; US20240091331; US20250325654; and U.S. patent application Ser. Nos. 19/356,950 and 19/357,163, and all priority applications referenced therein. The term comprising, wherever used, is intended to include the terms consisting and consisting essentially of. Furthermore, the terms comprising, including, containing and the like are not intended to be limiting. It is intended that the specification and examples be considered exemplary only with the true scope and spirit of the invention indicated by the following claims.

1. A composition containing two or more monoclonal antibodies:
wherein each of the two or more monoclonal antibodies are specifically reactive against a conserved region of an amino acid sequence of different viral proteins; and
wherein at least one of the two or more monoclonal antibodies have been altered to have an extended half-life as compared to an unaltered form of the at least one of the two or more monoclonal antibodies.

2. The composition of claim 1, wherein at least one of the two or more monoclonal antibodies are IgG antibodies.

3. The composition of claim 1, wherein at least one of the two or more monoclonal antibodies are IgM antibodies.

4. The composition of claim 1, wherein at least one of the two or more monoclonal antibodies are fully human or humanized antibodies.

5. The composition of claim 1, wherein at least one of the two or more monoclonal antibodies are bi-specific or multi-specific.

6. The composition of claim 1, wherein the different viral proteins are selected from the group consisting of influenza virus hemagglutinin protein (HA), neuraminidase protein (NA), matrix protein M1, matrix protein M2, and matrix protein M2e.

7. The composition of claim 6, containing three different monoclonal antibodies that are each specifically reactive against different amino acid sequences of hemagglutinin, neuraminidase, or matrix proteins of influenza virus.

8. The composition of claim 1, wherein the different viral proteins are selected from the group consisting of coronavirus spike protein(S), nucleocapsid protein (NC), matrix protein (M), envelope protein (E), and polymerase protein (P).

9. The composition of claim 1, wherein at least one of the two or more monoclonal antibodies is specifically reactive to the amino acid sequences of a single composite epitope of two similar viral virus epitopes.

10. The composition of claim 1, wherein at least one of the two or more monoclonal antibodies is specifically reactive to the amino acid sequences of a mimotope peptide, a fusion peptide, a peptide conjugate, or a synthetic peptide.

11. The composition of claim 1, wherein at least one of the two or more monoclonal antibodies have been altered by YTE modification, LS variants, or REW mutations.

12. The composition of claim 1, comprising a pharmaceutically acceptable carrier.

13. The composition of claim 12, wherein the pharmaceutically acceptable carrier comprises a chemical agent, diluent, or excipient comprises water, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, or a combination thereof.

14. A hybridoma that expresses at least one of the two or more monoclonal antibodies of claim 1.

15. A method of treatment of a patient comprising:
providing the composition of two or more antibodies of claim 1; and
administering the composition of antibodies to the patient.

16. A composition containing three or more different monoclonal antibodies, wherein:
at least one monoclonal antibody is specifically reactive to an epitope of hemagglutinin protein of influenza virus;
at least one monoclonal antibody is specifically reactive to an epitope of neuraminidase protein of influenza virus;
at least one monoclonal antibody is specifically reactive to an epitope of a matrix protein of influenza virus; and
one or more of the monoclonal antibodies have been altered to have an extended half-life as compared to an unaltered of the one or more monoclonal antibodies.

17. The composition of claim 16, wherein at least one of the three or more different monoclonal antibodies have been altered by YTE modification, LS variants, or REW mutations.

18. The composition of claim 16, wherein each of the three or more different monoclonal antibodies have been altered to have an extended half-life as compared to the unaltered three or more monoclonal antibodies.

19. The composition of claim 16, wherein each of the three or more different monoclonal antibodies are IgG antibodies.

20. The composition of claim 16, wherein each of the three or more different monoclonal antibodies are IgM antibodies.

21. The composition of claim 16, wherein each of the three or more different monoclonal antibodies are fully human or humanized antibodies.

22. A composition containing three or more different monoclonal antibodies, wherein:
at least one monoclonal antibody is specifically reactive to an epitope of a spike protein of coronavirus;
at least one monoclonal antibody is specifically reactive to an epitope of an envelope protein of coronavirus;
at least one monoclonal antibody is specifically reactive to an epitope of a matrix protein of coronavirus; and
one or more of the three or more monoclonal antibodies have been altered to have an extended half-life as compared to an unaltered of the one or more monoclonal antibodies.

23. The composition n of claim 22, wherein at least one of the three or more different monoclonal antibodies have been altered by YTE modification, LS variants, or REW mutations.

24. The composition of claim 22, wherein each of the three or more different monoclonal antibodies have been altered to have an extended half-life as compared to the unaltered forms of the three or more different monoclonal antibodies.

25. The composition of claim 22, wherein each of the three or more different monoclonal antibodies are IgG antibodies.

26. The composition of claim 22, wherein each of the three or more different monoclonal antibodies are IgM antibodies.

27. The composition of claim 22, wherein each of the three or more different monoclonal antibodies are fully human or humanized antibodies.

28. A method of preventing or treating a patient comprising:
providing the composition of three or more different monoclonal antibodies of claim 22; and
administering the composition of antibodies to the patient.

29. A monoclonal antibody that is specifically reactive to at least two different conserved regions of one or more different viral proteins; and
wherein the monoclonal antibody has been altered to have an extended half-life as compared to an unaltered form of the monoclonal antibody.

30. The monoclonal antibody of claim 29, which is an IgG or an IgM.

31. The monoclonal antibody of claim 29, which is partly or fully human or humanized.

32. The monoclonal antibody of claim 29, wherein the different viral proteins are selected from the group consisting of influenza virus hemagglutinin protein (HA), neuraminidase protein (NA), matrix protein M1, matrix protein M2, and matrix protein M2e.

33. The monoclonal antibody of claim 29, wherein the different viral proteins are selected from the group consisting of coronavirus spike protein(S), nucleocapsid protein (NC), matrix protein (M), envelope protein (E), and polymerase protein (P).

34. The monoclonal antibody of claim 29, which is specifically reactive to an epitope of a coronavirus protein and an epitope of an influenza virus protein.

35. The monoclonal antibody of claim 34, wherein the coronavirus epitope is a conserved region of a polymerase protein and the influenza virus epitope is a conserved region of a neuraminidase protein.

36. A method of preventing or treating a patient comprising:
providing the monoclonal antibody of claim 29; and
administering the monoclonal antibody to the patient.


SEQUENCE LISTING (143 sequences)

SEQ ID NO: 1 - protein, 19 aa, synthetic construct
DWSGYSGSFVQHPELTGLD

SEQ ID NO: 2 - protein, 7 aa, synthetic construct
ETPIRNE

SEQ ID NO: 3 - protein, 15 aa, synthetic construct
FVIREPFISCSHLEC

SEQ ID NO: 4 - protein, 6 aa, synthetic construct
GNFIAP

SEQ ID NO: 5 - protein, 6 aa, synthetic construct
GNLIAP

SEQ ID NO: 6 - protein, 7 aa, synthetic construct
GNLFIAP

SEQ ID NO: 7 - protein, 7 aa, synthetic construct
GNLIFAP

SEQ ID NO: 8 - protein, 8 aa, synthetic construct
HYEECSCY

SEQ ID NO: 9 - protein, 10 aa, synthetic construct
LLTEVETPIR

SEQ ID NO: 10 - protein, 11 aa, synthetic construct
LLTEVETPIRN

SEQ ID NO: 11 - protein, 12 aa, synthetic construct
LLTEVETPIRNE

SEQ ID NO: 12 - protein, 18 aa, synthetic construct
DWSGYSGSFVQHPELTGL

SEQ ID NO: 13 - protein, 9 aa, synthetic construct
EVETPIRNE

SEQ ID NO: 14 - protein, 39 aa, synthetic construct
FLLPEDETPIRNEWGLLTDDETPIRQYIKANSKFIGITE

SEQ ID NO: 15 - protein, 64 aa, synthetic construct
GNLFIAPGNLFIAPHYEECSCYHYEECSCYQYIKANSKFIGITEHYEECSCYTPIRNETPIRNE

SEQ ID NO: 16 - protein, 35 aa, synthetic construct
GNLFIAPGNLFIAPQYIKANSKFIGITEGNLFIAP

SEQ ID NO: 17 - protein, 48 aa, synthetic construct
HYEECSCYDWSGYSGSFVQHPELTGLHYEECSCYQYIKANSKFIGITE

SEQ ID NO: 18 - protein, 33 aa, synthetic construct
ITGFAPFSKDNSIRLSAGGDIWVTREPYVSCDP

SEQ ID NO: 19 - protein, 7 aa, synthetic construct
IWGIHHP

SEQ ID NO: 20 - protein, 7 aa, synthetic construct
IWGVHHP

SEQ ID NO: 21 - protein, 8 aa, synthetic construct
IWGVIHHP

SEQ ID NO: 22 - protein, 8 aa, synthetic construct
IWGIVHHP

SEQ ID NO: 23 - protein, 16 aa, synthetic construct
KSCINRCFYVELIRGR

SEQ ID NO: 24 - protein, 27 aa, synthetic construct
LLTEVETPIRNESLLTEVETPIRNEWG

SEQ ID NO: 25 - protein, 13 aa, synthetic construct
LLTEVETPIRNEW

SEQ ID NO: 26 - protein, 14 aa, synthetic construct
LLTEVETPIRNEWG

SEQ ID NO: 27 - protein, 11 aa, synthetic construct
LTEVETPIRNE

SEQ ID NO: 28 - protein, 12 aa, synthetic construct
LTEVETPIRNEW

SEQ ID NO: 29 - protein, 13 aa, synthetic construct
LTEVETPIRNEWG

SEQ ID NO: 30 - protein, 9 aa, synthetic construct
MSLLTEVET

SEQ ID NO: 31 - protein, 10 aa, synthetic construct
MSLLTEVETP

SEQ ID NO: 32 - protein, 11 aa, synthetic construct
MSLLTEVETPI

SEQ ID NO: 33 - protein, 12 aa, synthetic construct
MSLLTEVETPIR

SEQ ID NO: 34 - protein, 13 aa, synthetic construct
MSLLTEVETPIRN

SEQ ID NO: 35 - protein, 14 aa, synthetic construct
MSLLTEVETPIRNE

SEQ ID NO: 36 - protein, 20 aa, synthetic construct
MSLLTEVETPIRNETPIRNE

SEQ ID NO: 37 - protein, 15 aa, synthetic construct
MSLLTEVETPIRNEW

SEQ ID NO: 38 - protein, 16 aa, synthetic construct
MSLLTEVETPIRNEWG

SEQ ID NO: 39 - protein, 24 aa, synthetic construct
MSLLTEVETPIRNEWGCRCNDSSD

SEQ ID NO: 40 - protein, 8 aa, synthetic construct
SLLTEVET

SEQ ID NO: 41 - protein, 11 aa, synthetic construct
SLLTEVETPIR

SEQ ID NO: 42 - protein, 13 aa, synthetic construct
SLLTEVETPIRNE

SEQ ID NO: 43 - protein, 14 aa, synthetic construct
SLLTEVETPIRNEW

SEQ ID NO: 44 - protein, 15 aa, synthetic construct
SLLTEVETPIRNEWG

SEQ ID NO: 45 - protein, 21 aa, synthetic construct
SLLTEVETPIRNEWGTPIRNE

SEQ ID NO: 46 - protein, 27 aa, synthetic construct
SLLTEVETPIRNEWGTPIRNETPIRNE

SEQ ID NO: 47 - protein, 33 aa, synthetic construct
SLLTEVETPIRNEWGTPIRNETPIRNETPIRNE

SEQ ID NO: 48 - protein, 25 aa, synthetic construct
SLLTEVETPIRNEWGLLTEVETPIR

SEQ ID NO: 49 - protein, 10 aa, synthetic construct
TEVETPIRNE

SEQ ID NO: 50 - protein, 6 aa, synthetic construct
TPIRNE

SEQ ID NO: 51 - protein, 8 aa, synthetic construct
VETPIRNE

SEQ ID NO: 52 - protein, 53 aa, synthetic construct
VTREPYVSCDPKSCINRCFYVELIRGRVTREPYVSCDPWQYIKANSKFIGITE

SEQ ID NO: 53 - protein, 6 aa, synthetic construct
WGIHHP

SEQ ID NO: 54 - protein, 6 aa, synthetic construct
WGVHHP

SEQ ID NO: 55 - protein, 7 aa, synthetic construct
WGVIHHP

SEQ ID NO: 56 - protein, 7 aa, synthetic construct
WGIVHHP

SEQ ID NO: 57 - protein, 8 aa, synthetic construct
YIWGIHHP

SEQ ID NO: 58 - protein, 8 aa, synthetic construct
YIWGVHHP

SEQ ID NO: 59 - protein, 9 aa, synthetic construct
YIWGVIHHP

SEQ ID NO: 60 - protein, 9 aa, synthetic construct
YIWGIVHHP

SEQ ID NO: 61 - protein, 14 aa, synthetic construct
QYIKANSKFIGITE

SEQ ID NO: 62 - protein, 15 aa, synthetic construct
PIRNEWGCRCNDSSD

SEQ ID NO: 63 - protein, 22 aa, synthetic construct
GNLFIAPWGVIHHPHYEECSCY

SEQ ID NO: 64 - protein, 10 aa, synthetic construct
CAGAGNFIAP

SEQ ID NO: 65 - protein, 10 aa, synthetic construct
CAGAGNLIAP

SEQ ID NO: 66 - protein, 11 aa, synthetic construct
CAGAGNLFIAP

SEQ ID NO: 67 - protein, 10 aa, synthetic construct
CAGAWGVHHP

SEQ ID NO: 68 - protein, 10 aa, synthetic construct
CAGAWGIHHP

SEQ ID NO: 69 - protein, 11 aa, synthetic construct
CAGAWGVIHHP

SEQ ID NO: 70 - protein, 11 aa, synthetic construct
CAGAWGIVHHP

SEQ ID NO: 71 - protein, 13 aa, synthetic construct
GNLIAPWGVIHHP

SEQ ID NO: 72 - protein, 17 aa, synthetic construct
CAGAGNLIAPWGVIHHP

SEQ ID NO: 73 - protein, 14 aa, synthetic construct
GNLFIAPWGVIHHP

SEQ ID NO: 74 - protein, 18 aa, synthetic construct
CAGAGNLFIAPWGVIHHP

SEQ ID NO: 75 - protein, 8 aa, synthetic construct
HYEECSCY

SEQ ID NO: 76 - protein, 12 aa, synthetic construct
CAGAHYEECSCY

SEQ ID NO: 77 - protein, 26 aa, synthetic construct
CAGAGNLFIAPWGVIHHPHYEECSCY

SEQ ID NO: 78 - protein, 28 aa, synthetic construct
GNLFIAPWGVIHHPGNLFIAPWGVIHHP

SEQ ID NO: 79 - protein, 32 aa, synthetic construct
CAGAGNLFIAPWGVIHHPGNLFIAPWGVIHHP

SEQ ID NO: 80 - protein, 22 aa, synthetic construct
HYEECSCYGNLFIAPWGVIHHP

SEQ ID NO: 81 - protein, 22 aa, synthetic construct
GNLFIAPHYEECSCYWGVIHHP

SEQ ID NO: 82 - protein, 39 aa, synthetic construct
SLLTEVETPIRNEWGLLTEVETPIRQYIKANSKFIGITE

SEQ ID NO: 83 - protein, 35 aa, synthetic construct; identical to SEQ ID NO: 16

SEQ ID NO: 84 - protein, 48 aa, synthetic construct; identical to SEQ ID NO: 17

SEQ ID NO: 85 - protein, 52 aa, synthetic construct
VTREPYVSCDPKSCINRCFYVELIRGRVTREPYVSCDPQYIKANSKFIGITE

SEQ ID NO: 86 - protein, 18 aa, synthetic construct
DWSGYSGSFVQHPELTGL

SEQ ID NO: 87 - protein, 33 aa, synthetic construct; identical to SEQ ID NO: 18

SEQ ID NO: 88 - protein, 16 aa, synthetic construct
KSCINRCFYVELIRGR

SEQ ID NO: 89 - protein, 12 aa, synthetic construct
GNLFIAPRYAFA

SEQ ID NO: 90 - protein, 16 aa, synthetic construct
CAGAGNLFIAPRYAFA

SEQ ID NO: 91 - protein, 11 aa, synthetic construct
GNLVVPRYAFA

SEQ ID NO: 92 - protein, 15 aa, synthetic construct
CAGAGNLVVPRYAFA

SEQ ID NO: 93 - protein, 11 aa, synthetic construct
GNLIAPRYAFA

SEQ ID NO: 94 - protein, 15 aa, synthetic construct
CAGAGNLIAPRYAFA

SEQ ID NO: 95 - protein, 6 aa, synthetic construct
GNLVVP

SEQ ID NO: 96 - protein, 10 aa, synthetic construct
CAGAGNLVVP

SEQ ID NO: 97 - protein, 15 aa, synthetic construct
FVIREPFISCSHLEC

SEQ ID NO: 98 - protein, 19 aa, synthetic construct
CAGAFVIREPFISCSHLEC

SEQ ID NO: 99 - protein, 22 aa, synthetic construct
GNLFIAPWGVIHHPHYEECSCY

SEQ ID NO: 100 - protein, 36 aa, synthetic construct
GNLFIAPWGVIHHPHYEECSCYQYIKANSKFIGITE

SEQ ID NO: 101 - protein, 36 aa, synthetic construct
QYIKANSKFIGITEGNLFIAPWGVIHHPHYEECSCY

SEQ ID NO: 102 - protein, 7 aa, synthetic construct
HVEECSY

SEQ ID NO: 103 - protein, 6 aa, synthetic construct
WFIHHP

SEQ ID NO: 104 - protein, 11 aa, synthetic construct
DLWSYNAELLV

SEQ ID NO: 105 - protein, 11 aa, synthetic construct
DIWTYNAELLV

SEQ ID NO: 106 - protein, 30 aa, synthetic construct
ARDLICAQKWPWYIWLGFIAGLENQKLIAN

SEQ ID NO: 107 - protein, 16 aa, synthetic construct
ENQKLIANARDLICAQ

SEQ ID NO: 108 - protein, 14 aa, synthetic construct
QYIKANSKFIGITE

SEQ ID NO: 109 - protein, 22 aa, synthetic construct
GNLFIAPWGVIHHPHYEECSCY

SEQ ID NO: 110 - protein, 7 aa, synthetic construct
GNLFIAP

SEQ ID NO: 111 - protein, 7 aa, synthetic construct
WGVIHHP

SEQ ID NO: 112 - protein, 8 aa, synthetic construct
HYEECSCY

SEQ ID NO: 113 - protein, 7 aa, synthetic construct
ETPIRNE

SEQ ID NO: 114 - protein, 10 aa, synthetic construct
TEVETPIRNE

SEQ ID NO: 115 - protein, 25 aa, synthetic construct
SLLTEVETPIRNEWGLLTEVETPIR

SEQ ID NO: 116 - protein, 22 aa, synthetic construct
GNLFIAPWGVIHHPHYEECSCY

SEQ ID NO: 117 - protein, 46 aa, synthetic construct
GNLFIAPWGVIHHPHYEECSCYTEVETPIRNEQYIKANSKFIGITE

SEQ ID NO: 118 - protein, 46 aa, synthetic construct
QYIKANSKFIGITEGNLFIAPWGVIHHPHYEECSCYTEVETPIRNE

SEQ ID NO: 119 - protein, 7 aa, synthetic construct
YPKCDRA

SEQ ID NO: 120 - protein, 9 aa, synthetic construct
WDYPKCDRA

SEQ ID NO: 121 - protein, 23 aa, synthetic construct
SLDQINVTFLDLEYEMKKLEESY

SEQ ID NO: 122 - protein, 23 aa, synthetic construct
SLDQINVTFLDLEYEMKKLEESY

SEQ ID NO: 123 - protein, 37 aa, synthetic construct
SLDQINVTFLDLEYEMKKLEESYQYIKANSKFIGITE

SEQ ID NO: 124 - protein, 9 aa, synthetic construct
WDYPKCDRA

SEQ ID NO: 125 - protein, 23 aa, synthetic construct
WDYPKCDRAQYIKANSKFIGITE

SEQ ID NO: 126 - protein, 46 aa, synthetic construct
WDYPKCDRASLDQINVTFLDLEYEMKKLEESYQYIKANSKFIGITE

SEQ ID NO: 127 - protein, 41 aa, synthetic construct
WDYPKCDRATEVETPIRNEHYEECSCYQYIKANSKFIGITE

SEQ ID NO: 128 - protein, 37 aa, synthetic construct; identical to SEQ ID NO: 123

SEQ ID NO: 129 - protein, 9 aa, synthetic construct
WDYPKCDRA

SEQ ID NO: 130 - protein, 7 aa, synthetic construct
YPKCDRA

SEQ ID NO: 131 - protein, 8 aa, synthetic construct
ARDLICAQ

SEQ ID NO: 132 - protein, 14 aa, synthetic construct
KWPWYIWLGFIAGL

SEQ ID NO: 133 - protein, 8 aa, synthetic construct
ENQKLIAN

SEQ ID NO: 134 - protein, 30 aa, synthetic construct; identical to SEQ ID NO: 106

SEQ ID NO: 135 - protein, 16 aa, synthetic construct
ENQKLIANARDLICAQ

SEQ ID NO: 136 - protein, 25 aa, synthetic construct
WDYPKCDRAENQKLIANARDLICAQ

SEQ ID NO: 137 - protein, 31 aa, synthetic construct
WDYPKCDRAENQKLIANKWPWYIWLGFIAGL

SEQ ID NO: 138 - protein, 39 aa, synthetic construct
ARDLICAQENQKLIANWDYPKCDRAQYIKANSKFIGITE

SEQ ID NO: 139 - protein, 76 aa, synthetic construct
KWPWYIWLGFIAGLWDYPKCDRAQYIKANSKFIGITEARDLICAQENQKLIANWDYPKCDRAQYIKANSKFIGITE

SEQ ID NO: 140 - protein, 69 aa, synthetic construct
ARDLICAQENQKLIANQYIKANSKFIGITEARDLICAQENQKLIANWDYPKCDRAQYIKANSKFIGITE

SEQ ID NO: 141 - protein, 80 aa, synthetic construct
WDYPKCDRATEVETPIRNEHYEECSCYQYIKANSKFIGITEARDLICAQENQKLIANWDYPKCDRAQYIKANSKFIGITE

SEQ ID NO: 142 - protein, 39 aa, synthetic construct
HYEECSCYWDYPKCDRAVETPIRNEQYIKANSKFIGITE

SEQ ID NO: 143 - protein, 40 aa, synthetic construct
ENQKLIANTEVETPIRNEHYEECSCYQYIKANSKFIGITE